(19) United States
(12) Patent Application Publication
(10) Pub. No.: US 2005/0004071 A1
(43) Pub. Date: Jan. 6, 2005

(54) Charged polysaccharides resistant to lysosomal degradation during kidney filtration and renal passage and their use to treat or prevent infection by coronaviruses

(76) Inventor: Wayne D. Comper, North Ringwood (AU)

Correspondence Address: JONES DAY, 222 EAST 41ST ST, NEW YORK, NY, 10017, US

(21) Appl. No.: 10/827,757

(22) Filed: Apr. 20, 2004

Related U.S. Application Data
(60) Provisional application No. 60/464,294, filed on Apr. 21, 2003.

(51) Int. Cl. A61K 31/737

(52) U.S. Cl. 514/54

(57) ABSTRACT

This invention relates to methods for treating, preventing or managing coronavirus infections in mammals using sulfated polysaccharides. More particularly, this invention relates to methods of treating, preventing or managing infection by coronaviruses, particularly viral infections leading to or causing diseases such as the newly discovered Severe Acute Respiratory Syndrome (“SARS”). The invention involves the use of sulfated polysaccharides which are abundant, non-toxic and inexpensive and which are potent antivirals in vivo.

This application claims the benefit of U.S. Provisional Application No. 60/464,294, filed Apr. 21, 2003, which is incorporated herein by reference

### 1. FIELD OF THE INVENTION

This invention relates to methods for treating, preventing or managing coronavirus infections in mammals using sulfated polysaccharides. More particularly, this invention relates to methods of treating, preventing or managing infection by coronaviruses, particularly viral infections leading to or causing diseases such as the newly discovered Severe Acute Respiratory Syndrome (“SARS”). The invention involves the use of sulfated polysaccharides which are abundant, non-toxic and inexpensive and which are potent antivirals in vivo.

### 2. BACKGROUND OF THE INVENTION

Charged polysaccharides, particularly sulfated polysaccharides, have demonstrated potent antimicrobial activities in vitro. (Baba et al., Antiviral Res 9:335-343, 1988; Ito et al., Antiviral Res. 7(36):1-367, 1987). For example, sulfated polysaccharides such as dextran sulfate, heparin, and pentosan polysulfate have been reported to be potent inhibitors of HIV, paramyxoviruses, cytomegaloviruses, influenza viruses, semlikiviruses (Lüscher-Mattli et al., Arch Virol 130:317-326, 1993) and herpes simplex viruses in vitro (Baba et al., Antimicrob. Agents Chemotherapy 32:1742-45, 1988; Pancheva, Antiviral Chem Chemotherapy 4:189-191, 1993). However, these known compounds have disappointingly poor activity in vivo.

Dextran sulfate and heparin were first reported to inhibit HIV replication in vitro by Ito et al., Antiviral Res. 7:36 1-367, 1987, Deringer et al. (U.S. Pat. No. 5,153,181) and Ueno and Kuno, Lancet 2:796-97, 1987. Later, several other sulfated polysaccharides were shown to inhibit HIV replication at concentrations believed to be below their respective cytotoxicity thresholds, e.g., pentosan sulfate (Baba et al., Antiviral Res 9: 335-343, 1988; Biesert et al., Aids 2(6):449-57, 1988), fuciodan (Baba et al., Antiviral Res 9:335-343, 1988), lambda-, kappa- and iota-carrageenan (Baba et al., Antiviral Res 9: 335-343, 1988), lentinan sulfate (Yoshida et al., Biochem. Pharmacol. 37(15):2887-91, 1988), mannan sulfate (Ito et al., Eur. J. Clin. Microbiol. Infect. Dis. 8: 191-193, 1989), dextrin sulfate (Ito et al. Antiviral Chem. Chemother., 2:41-44, 1991), sulfoeveman (Weiler et al., J Gen Virol 71:1957-1963, 1990), and sulfated cyclodextrins (Schols et al., J Acquired Immune Def. Syndr 4:677-85,1991.).

Conventional or commercial dextran sulfate has a percent of sulfation of about 17-22%. It is widely accepted that increasing sulfur content increases activity of this material. For example, increasing sulfur content increases anti-coagulant activity. (Hirata et al., Biosci. Biotech. Biochem. 58(2):406-407, 1994). Similarly, it is widely accepted that increasing the sulfur content of sulfated polysaccharides increases their in vitro antiviral activity. See, e.g., Witvrouw et al., General Pharmacology 29 (4): 497-512, 1997; Nakashima et al., Jpn. J. Cancer Res. (Gann) 78:1164-68, 1987; and Baba et al., J. AIDS 493-499, 1990. Again, these studies have demonstrated a marked increase in the in vitro activity of sulfated polysaccharides with the increase in sulfation, although the lack of in vivo efficacy remains. (Mathis et al., Antimicrobial Agents & Chemotherapy 2147-2150, 1991; Flexner et al. Antimicrob Agents Chemotherapy 35:2544-2550, 1991; Abrams et al., Annals of Internal Medicine 110: 183-188 (1989); Hiebert et al., J. Lab & Clin. Med. 133:161-170 (1999)) Indeed, lack of in vivo efficacy and the in vivo toxicity of compounds with a high degree of sulfation has been an unsolvable problem to date.

Dextran sulfate is a heparin-like polysaccharide containing approximately 17% sulfur with up to three sulfate groups per glucose molecule. Merck Index, 12^{th} ed., Whitehouse Station: Merck, 1996. According to British Pharmacopeia (circa 1958), dextran sulfate can be formulated as a sterile solution for injection. Prior to the present invention, dextran sulfate has been shown to have anticoagulant properties and antihyperlipoprteinemic properties in addition to the anti-HIV properties described above. (Ricketts, Biochem J. 51, 129 (1952)).

The Center for Disease Control and Prevention (“CDC”) is investigating a new disease called severe acute respiratory syndrome (“SARS”). The disease was first reported among people in Guangdong Province, China, Hanoi, Vietnam and Hong Kong. SARS is believed to be a virus which is spread, at least, by close contact between people. The CDC has sequenced the genome for the virus believed to be responsible for the global epidemic of SARS. The sequence data confirms that SARS is caused by a coronavirus which is previously unrecognized. The majority of the sequence was derived directly from viral RNA. The genome of the SARS coronavirus is 29,727 nucleotides. See, <URL: www.cdc.gov/ncidod/SARS/sequence.htm> and generally Lee et al., 2003, “A Major Outbreak of Severe Acute Respiratory Syndrome in Hong Kong”, New England Journal of Medicine Apr. 7, 2003:epub ahead of print; Drosten et al., 2003, “Identification of a Novel Coronavirus in Patients with Severe Acute Respiratory Syndrome”, New England Journal of Medicine Apr. 10, 2003:epub ahead of print; Ksiazek et al., 2003, “A Novel Coronavirus Associated with Severe Acute Respiratory Syndrome”, New England Journal of Medicine Apr. 10, 2003:epub ahead of print). Thus, there is a need for antivirals effective against coronaviruses, particularly the coronavirus responsible for SARS.

### 3. SUMMARY OF THE INVENTION

The invention encompasses novel methods for the treatment or prevention of coronavirus infection, and novel pharmaceutical compositions which utilize sulfated polysaccharides, particularly dextran sulfates as antiviral against coronavirus infection, particularly respiratory infection or viral infection leading to respiratory disease. The invention encompasses in a preferred embodiment the use of sulfated polysaccharides, having a percent of sulfur with respect to the simple sugar residue of greater than 2% and less than 20%, more preferably greater than 3% and less than 13%, within the methods and compositions of the invention. The sulfated polysaccharides are most preferably sulfated dextrans having an α-1,6-glycosidic linkage.

The invention further encompasses the use of sulfated polysaccharides having a molecular weight between 500 and 1,000,000, preferably above 5,000; more preferably above 25,000; most preferably above 40,000 within the methods and compositions. Ranges of 5,000 to 1,000,000, 25,000 to 500,000 and 40,000 to 300,000 are also encompassed by the invention for oral or parenteral use. However, for topical administration, the sulfated polysaccharide may have a molecular weight higher than 500,000 in a preferred embodiment. In an alternative embodiment, the composition has only about 10% variability in the molecular weight and preferably about 5% variation.

In addition to dextran sulfates, the sulfated polysaccharide can be cellulose sulfate, dextrin sulfate, cyclodextrin, or one of the other materials found in Table 1 below, preferably wherein the percent of sulfur is within the range of 2% to 20%, more preferably 3% to 13%. Moreover, substituted polysaccharides such as carboxymethyl substituted or periodated treated polysaccharides, particularly substituted dextran sulfates such as carboxymethyl substituted dextran sulfate or periodate treated dextran sulfates can be used. In one embodiment, the sulfated polysaccharide is homogenous with respect to molecular weight, percent of sulfation or both.

In one aspect, the invention encompasses a method for treating, preventing or managing coronavirus infection comprising administering a therapeutically effective amount of a sulfated polysaccharide or salt thereof into the blood stream, or lymphatic system of a mammal in need thereof. Preferably, the percent of sulfur of the polysaccharide is effective to enable maximal interaction of constituent sulfate groups with the virus which causes the coronaviral infection, and wherein the sulfated polysaccharide is not substantially endocytosed or degraded by cell receptor binding in the mammal, and thereby retains antiviral activity in vivo. In order words, the preferred sulfated polysaccharides of the invention, in contrast to those previously reported in the art, are active and relatively non-toxic in vivo as result of their degree of sulfation.

In a preferred embodiment the method is for treating, preventing or managing a viral infection, including but not limited to enveloped viruses whether DNA or RNA viruses. In a separate and preferred method the viruses to be treated include but are not limited to positive-sense single-stranded RNA viruses, particularly coronaviruses.

The invention also encompasses pharmaceutical compositions suitable for parenteral administration to a patient comprising a therapeutically or pharmaceutically acceptable amount of a polysaccharide of the invention; pharmaceutical compositions suitable for oral administration to a patient comprising a therapeutically or pharmaceutically acceptable amount of a polysaccharide of the invention; and pharmaceutical compositions suitable for topical administration to a patient comprising a therapeutically or pharmaceutically acceptable amount of a polysaccharide of the invention, preferably having a molecular weight greater than 500,000. In a preferred embodiment, each of these compositions is in single unit dosage form and comprising an amount of active ingredient sufficient to treat or prevent human infection by coronavirus, particularly infection causing or leading to respiratory disease.

It should be noted that the invention also encompasses the use of the sulfated polysaccharides of the invention as disinfectants that can be used to disinfect inanimate objects particularly in hospitals, laboratories, lavatories, auditoriums, stadiums, convention centers, restaurants, fitness centers, subway terminals, bus terminals, airports, post offices, offices, sewage treatment facilities, sewers, water treatment facilities, pumping stations, automobiles, airplanes, trains, homes, lockers, and furniture to prevent the spread of coronavirus. The invention also encompasses disinfectant compositions such as solutions, gels, powders, concentrates, lotions, creams, sprays, soaps, or foams comprising one or more of the sulfated polysaccharides described herein.

The viral infections encompassed by the methods of the invention, particularly the specific viruses to be treated and specific sulfated dextrans to be used, are described in detail below.

3.1 Definitions

As used herein, the term “patient” or “subject” means an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In certain embodiments, the patient is an infant, child, adolescent, adult or geriatric patient. In addition, the patient includes immunocompromised patients such as HIV positive patients, cancer patients, patients undergoing immunotherapy or chemotherapy. In a particular embodiment, the patient is a healthy individual, i.e., not displaying symptoms of other viral infections.

As used herein, a “therapeutically effective amount” refers to an amount of the sulfated polysaccharide of the invention sufficient to provide a benefit in the treatment or management of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

As used herein, a “prophylactically effective amount” refers to an amount of a compound of the invention or other active ingredient sufficient to result in the prevention of infection, recurrence or spread of viral infection. A prophylactically effective amount may refer to an amount sufficient to prevent initial infection or the recurrence or spread of the infection or a disease associated with the infection. Used in connection with an amount of a compound of the invention, the term preferably encompasses a non-toxic amount that improves overall prophylaxis or enhances the prophylactic efficacy of or synergies with another prophylactic or therapeutic agent.

As used herein, “in combination” refers to the use of more than one prophylactic and/or therapeutic agents simultaneously or sequentially and in a manner that their respective effects are additive or synergistic.

As used herein, the terms “manage”, “managing”, and “management” refer to the slowing or preventing the progression or worsening of the viral infection, reducing the viral load, or preventing the death or serious symptoms or effects associated with viral infection.

As used herein, the terms “prevent”, “preventing” and “prevention” refer to the prevention of the onset, recurrence, or spread of infection in a subject resulting from the administration of an active ingredient before the infection occurs.

As used herein, the terms “treat”, “treating” and “treatment” refer to the eradication or amelioration of the infection itself, causes of the infection, or symptoms associated therewith. In certain embodiments, such terms refer to minimizing the spread or worsening of the infection resulting from the administration of one or more prophylactic or therapeutic agents to a subject with such an infection.

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compound of the present invention include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

As used herein and unless otherwise indicated, the term “optically pure” or “stereomerically pure” means a composition that comprises one stereoisomer of a compound and is substantially free of other stereoisomers of that compound. For example, a stereomerically pure a compound having one chiral center will be substantially free of the opposite enantiomer of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. Since the compounds of the invention are primarily polysaccharides made of saccharides which can exist in either the D or L forms, the invention encompasses either or both D and L sugars. As such, for example, a stereomerically pure D sugar will be substantially free of the L form. In an alternative embodiment, the use of L forms of sulfated dextrans permits the use of a broader controlled range of sulfation from above 6% to about 20%. Thus, the methods and compositions disclosed herein include in an alternative embodiment the use of such levorotatory sugars or polymers made therefrom.

As used herein, the term “sulfated polysaccharide” means a sulfated material having more than ten units of simple sugar. Preferably the sulfated polysaccharide is an alpha(1,6) linked polysaccharide. The sulfated polysaccharides of the invention also preferably have a percent of sulfur that is sufficient for both in vitro and in vivo activity without significant toxicity.

As used herein, the term “dextran” means a polysaccharide containing a backbone of D-glucose units linked predominantly α-D(1,6), composed exclusively of α-D-glucopyranosyl units differing only in degree of branching and chain length.

As used herein, the term “dextran sulfate sodium” or “dextran sulfate”, “conventional dextran sulfate”, or “commercial dextran sulfate” unless otherwise qualified means a α-1,6-polyglucose containing approximately 17% sulfur with up to three sulfate groups per glucose molecule of varying molecular weight ranges, e.g., 4,000-500,000 Da.

As used herein, the terms “percent sulfation”, “percent of sulfation”, “percent of sulfate substitution” or “sulfation” means the percent of sulfur by molecular weight with respect to each simple sugar residue within the polysaccharide in question, optionally including a counterion, e.g., molecular weight of sulfation in the composition/total weight. In a preferred embodiment, the percent of sulfur is calculated as the percent of sulfur by molecular weight with respect to the sulfated sugar residue within the polysaccharide in question with sodium as the counterion. The percent of sulfation can be determined by elemental analysis of material which has been dialyzed to remove free sulfur, preferably of moisture/volatile free material dried in vacuo at 60° C. to a constant weight. Other methods of determining percent of sulfation are via moisture content analysis and titration. Sulfation is to be distinguished from “degree of substitution” or “equivalents” which is a measure of the number of sulfate groups per sugar moiety. However, it will be recognized by one of skill in the art that percent sulfation can be converted to a degree of substitution or equivalents and vice versa.

As used herein, the term “co-charged dextran polyanions” is dextran substituted to varying degrees with any combination of carboxymethyl groups, sulfate groups and sulfonate groups.

As used herein, the term “periodate treated anionic polysaccharides” means any anionic polysaccharide that has been treated with periodate to open the sugar ring without depolymerization or to otherwise increase the flexibility of the polysaccharide in order to increase interaction with the microbe.

### 4. BRIEF DESCRIPTION OF THE FIGURE

FIG. 1: Genomic nucleic acid sequence of SARS-associated coronavirus (Urbani strain) (SEQ ID.1).

### 5. DETAILED DESCRIPTION OF THE INVENTION

In one embodiment of the invention, the invention encompasses sulfated polysaccharides such as conventional dextran sulfate or variants thereof (e.g., dextran sulfate with a percent of sulfur that differs from the conventional material) that can be used to treat or prevent coronavirus infection including treatment or prevention of the recently identified coronavirus that leads to Severe Acute Respiratory Syndrome (“SARS”). In one embodiment, the sulfated polysaccharide has a percent of sulfation greater than 2% but below 20% range, and in a preferred embodiment greater that 6% but below 17%. The most preferred compositions or methods of the invention utilize sulfated α-1,6-linked polysaccharides or sulfated dextrans having the desired percent of sulfation and/or molecular weight which are flexible and thus useful against a wide variety of coronaviruses. In a most preferred embodiment, the range of percent sulfation is effective to enable maximal interaction of constituent sulfate groups with the coronavirus which causes the infection, and wherein the sulfated polysaccharide is not substantially endocytosed or degraded by cell receptor binding in the mammal, and thereby retains antiviral activity in vivo.

The present invention encompasses methods for treating, preventing or managing coronavirus infections in vivo, with a sulfated polysaccharide or a pharmaceutically acceptable salt thereof, having flexibility in its structure, a controlled degree of sulfation, preferably a low degree of sulfation as compared to conventional dextran sulfate.

The present invention also provides methods for the treatment, prevention, or management of SARS comprising administering to a patient in need thereof a therapeutically or prophylactically effective amount of a sulfated polysaccharide or pharmaceutically acceptable salts thereof.

Again, in a preferred embodiment, the sulfated polysaccharides used in the methods or compositions of the invention have a percent sulfation sufficient for in vivo anti-viral activity, but which is controlled to enable the compound to escape binding by cell receptors for high charge density polyanions and desulfation after passage through the kidney. This results in retention of anti-viral activity in vivo without toxicity or adverse effects.

Without being limited by any particular theory, the inventor believes that there is a range of charge density for sulfated polysaccharides within which they exhibit anti-viral activity in vitro and retain their anti-viral activity in vivo. In a preferred embodiment of the invention, the sulfated polysaccharides of the invention have a percent of sulfation of greater than 2% and below 20%, preferably greater than about 3% and below 17%, more preferably greater than about 4%, 5%, 6%, 7%, 8%, 9%, 10%, 11%, 12% or 13%, most preferably 9%, 9.5%, 10%, 10.5%, 11%, 11.5%, 12%, 12.2%, 12.5% or 12.8%.

A preferred sulfated polysaccharide used in the methods of the invention is sulfated dextran, or an α-1,6-linked polysaccharide, such as conventional dextran sulfate or a variant of dextran sulfate which has been modified to have the desired percent of sulfation. The sulfated dextran of the invention contain less than 20%, and may contain less than 19%, less than 18%, less than 17%, less than 16%, less than 15%, less than 14%, less than 13%, less then 12%, less than 11%, less than about 10%, less than 9%, less than 8%, and less than 7% sulfur, but more than 2% sulfur. In a preferred embodiment, the sulfated dextran variant has a sulfation of less than 13% and greater than 6%, more preferably, from about 7.0% to about 12.8%, even more preferably from about 8.5% to about 12.8%, and most preferably, from about 9.5% to less than 13%. Sulfated dextran having sulfation of about 12.2% and about 12.5% are particularly effective against SARS associated coronaviruses infections.

The sulfated polysaccharides of the invention, particularly the sulfated dextrans, can be prepared using known synthetic techniques and reagents. Several methods which are known in the art may be modified if the degree of sulfation is to be lowered. These methods include those described in Examples 1 and 3. The inventor has synthesized sulfated dextran with controlled sulphur contents and controlled degrees of sulfate substitution so that they are not taken up by cell receptors for highly charged polysaccharides. These polysaccharides exhibit essentially the same high antiviral activity in vivo as they do in vitro and have enhanced stability and longevity in vivo, as they are not readily taken up by cells they are also less toxic. Sulfated dextran, with controlled sulphur content is particularly suited as a viral cell attachment inhibitor because of its unique structure of essentially linear chain composed of an α-1,6-glycosidic linkage makes the most flexible of all polysaccharide chains that then enables maximal interaction of its constituent sulfate groups with positive charges on proteins of the virus and that it does not bind significantly to plasma proteins including albumin.

In another alternative embodiment, the invention encompasses the use of homogeneous sulfated polysaccharides. That is to say the sulfated polysaccharides administered in accordance with the methods described herein or utilized in the pharmaceutical compositions and dosage forms exhibit substantially the same percent of sulfation or molecular weight or both.

In a separate embodiment, the invention encompasses a method of treating, preventing or managing a respiratory infection in a mammal, which infection is caused by a coronavirus, comprising administering to a mammal in need thereof a therapeutically effective amount of a composition comprising a sulfated polysaccharide having a percent of sulfate substitution per glucose residue in the polysaccharide ranging from greater than 2% to less than 20%. Preferably the methods employ a compound wherein the range of percent sulfation is effective to enable maximal interaction of constituent sulfate groups with the virus which causes the infection, and wherein the sulfated polysaccharide is not substantially endocytosed or degraded by cell receptor binding in the mammal, and thereby retains antiviral activity in vivo. Preferably, the sulfated polysaccharide is sulfated dextran.

The invention further encompasses a method of treating, preventing or managing a coronavirus infection, particularly an infection leading to or causing respiratory disease including SARS, in a mammal which comprises administering to a mammal in need thereof an effective amount of a sulfated polysaccharide; preferably one having a percent of sulfation from about 2% to about 20%; more preferably from about 6% to about 13%; and most preferably from about 6% to about 13%.

In a further embodiment, the invention encompasses a method of treating, preventing or managing a coronavirus infection, particularly an infection leading to or causing respiratory disease including SARS, in a mammal which comprises administering to a mammal in need thereof an effective amount of a periodate-treated anionic polysaccharide. Preferably, the periodate treated anionic polysaccharide is a periodate treated sulfated dextran.

In another embodiment of the invention, the invention encompasses a method of treating, preventing or managing a coronavirus infection, particularly an infection leading to or causing respiratory disease including SARS, in a mammal which comprises administering to a mammal in need of such treatment or prevention an effective amount of a co-charged anionic polysaccharide which has a percent of sulfation which enables maximal interaction with the virus and which is not substantially endocytosed or degraded by cell receptor binding in the mammal thereby retaining antiviral activity in vivo. In a preferred embodiment, the co-charged anionic polysaccharide is co-charged with carboxymethyl groups, sulfonate groups, sulfate groups or mixtures thereof, more preferably the co-charged anionic polysaccharide is co-charged with carboxymethyl groups. In a specific embodiment, the co-charged anionic polysaccharide is carboxymethyl dextran sulfate or carboxymethyl cellulose.

5.1 Coronavirus Infections

Viral infections which can be treated, prevented or managed by the methods and compositions of the present invention include, but are not limited to, enveloped DNA and RNA viruses, more particularly, enveloped positive-sense single-stranded RNA viruses, and most particularly, viruses in the family Coronaviridae (or “coronaviruses”). As used herein, the term “coronavirus” refers to viruses in the family Coronaviridae, including those viruses in the genuses Coronavirus (including, e.g., those viruses in serologically distinct antigenic Groups I, II, and III) and Torovirus. Also included are viruses in the “floating genus” Arterivirus which have genome organization and replication strategy closely resembling those viruses in the Coronaviridae family.

In one embodiment, coronaviruses, which can be treated, prevented, or managed by methods of the present invention, include, but not limited to, berne virus, breda virus, infectious bronchitis virus (fowl), turkey bluecomb virus, transmissible gastroenteritis virus (swine), hemagglutinating encephalomyelitis virus (swine), porcine epidemic diarrhea virus, calf coronavirus, feline infectious peritonitis virus, feline enteric corona virus, canine coronavirus, mouse hepatitis viruses, rat coronavirus (sialodacryoadentis virus), rabbit coronavirus, bovine respiratory torovirus, porcine torovirus, feline torovirus, equine arteritis virus, lactate dehydrogenase-elevating virus (mice), simian hemorrhagic fever virus, Lelystad virus (porcine reproductive and respiratory syndrome virus), and VR2332 virus (swine). In a preferred embodiment, the coronavirus treated, prevented, or managed by methods of the present invention is not transmissible gastroenteritis virus. In another preferred embodiment, the coronavirus treated, prevented, or managed by methods of the present invention is not feline infectious peritonitis virus.

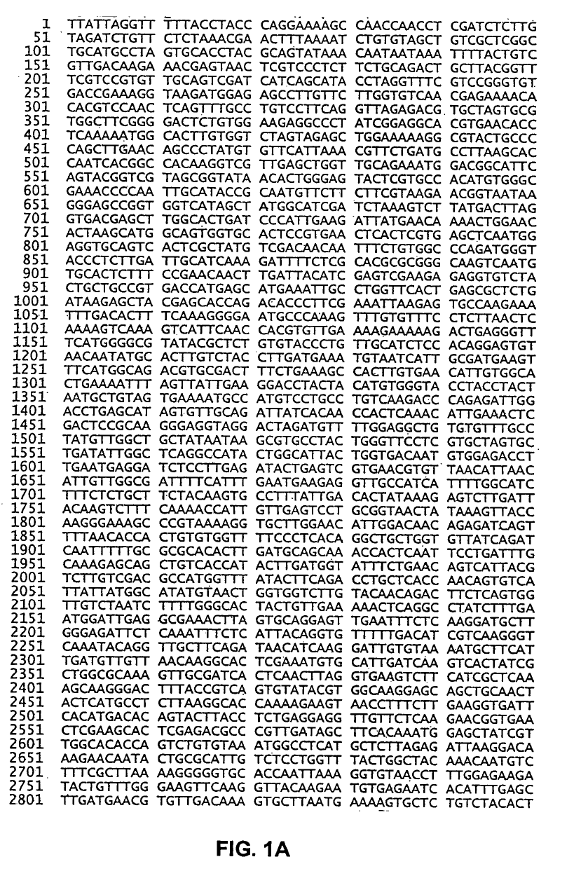

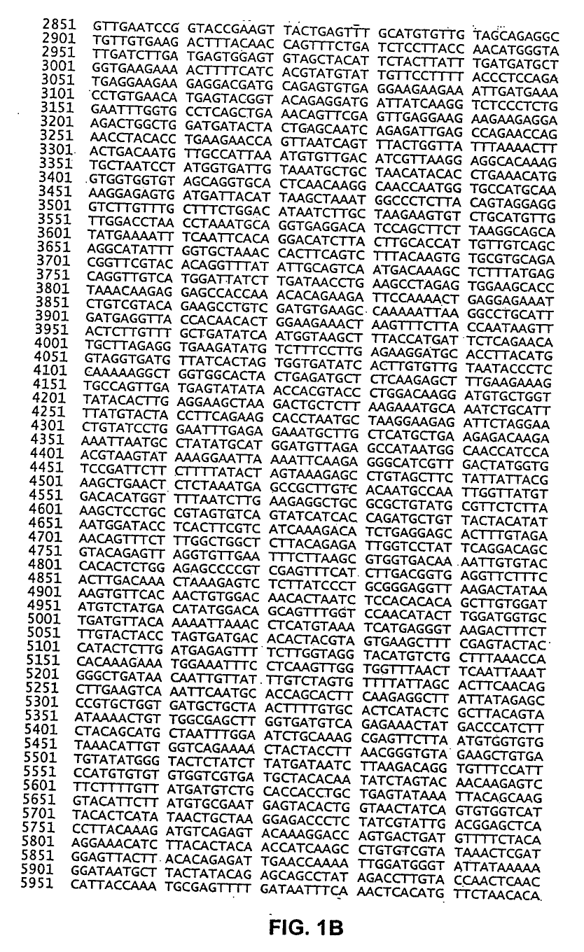

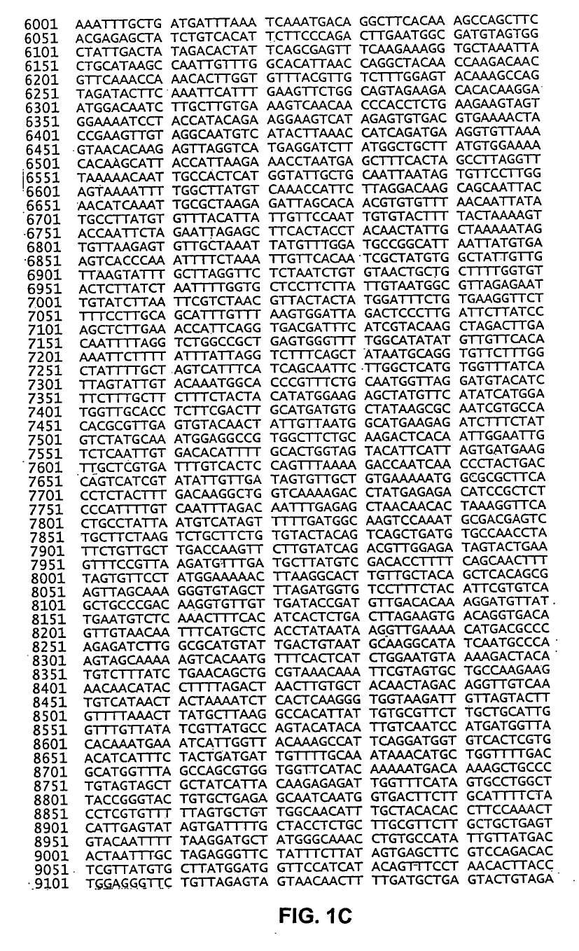

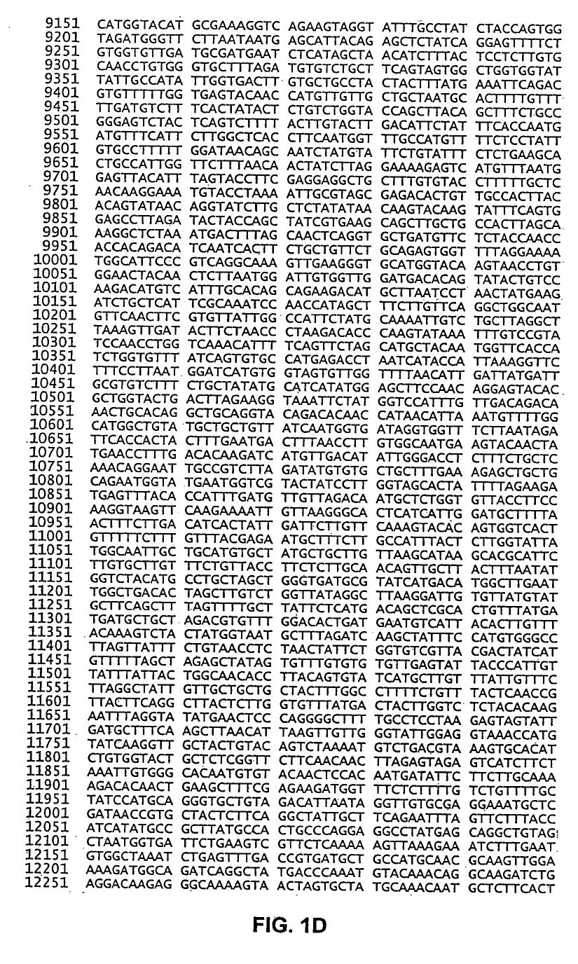

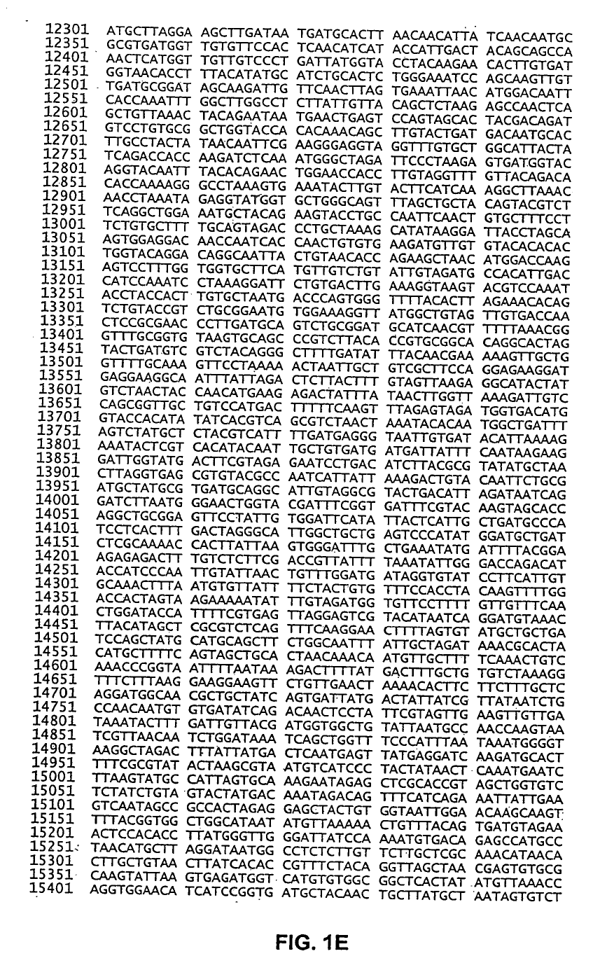

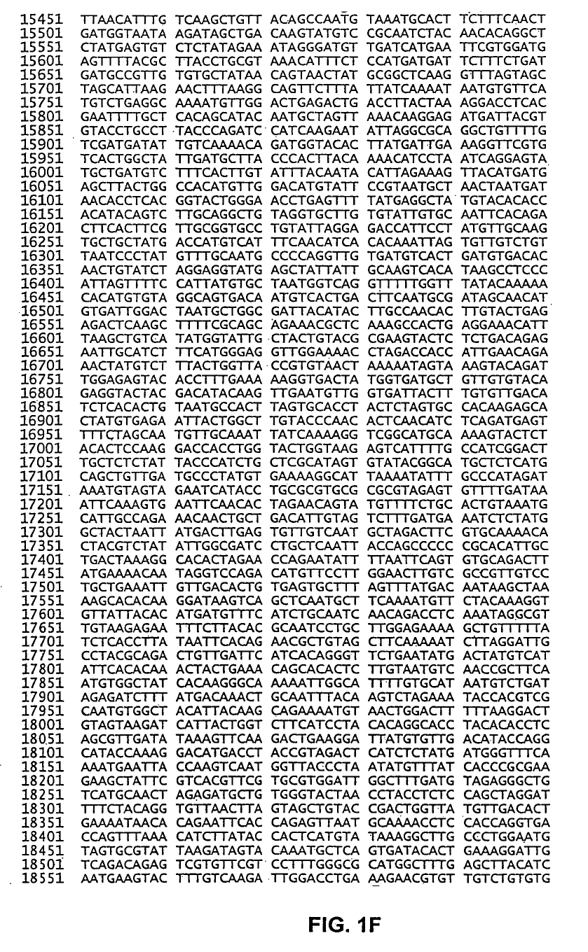

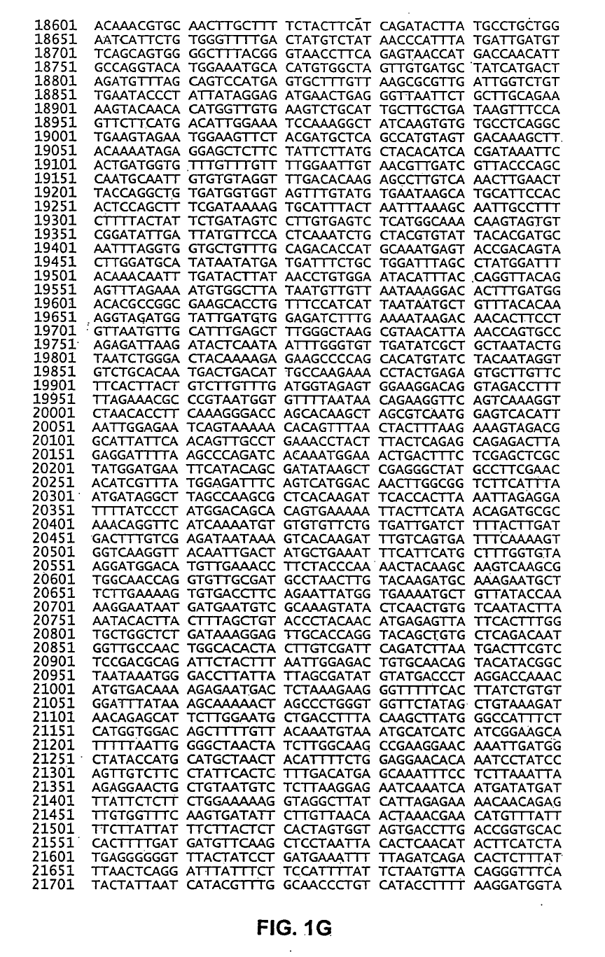

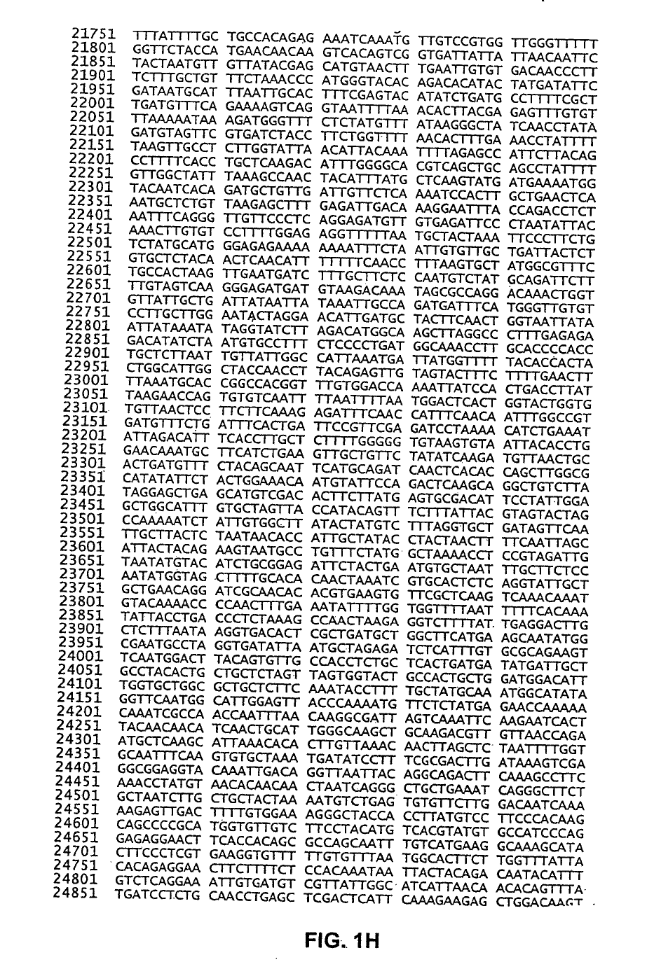

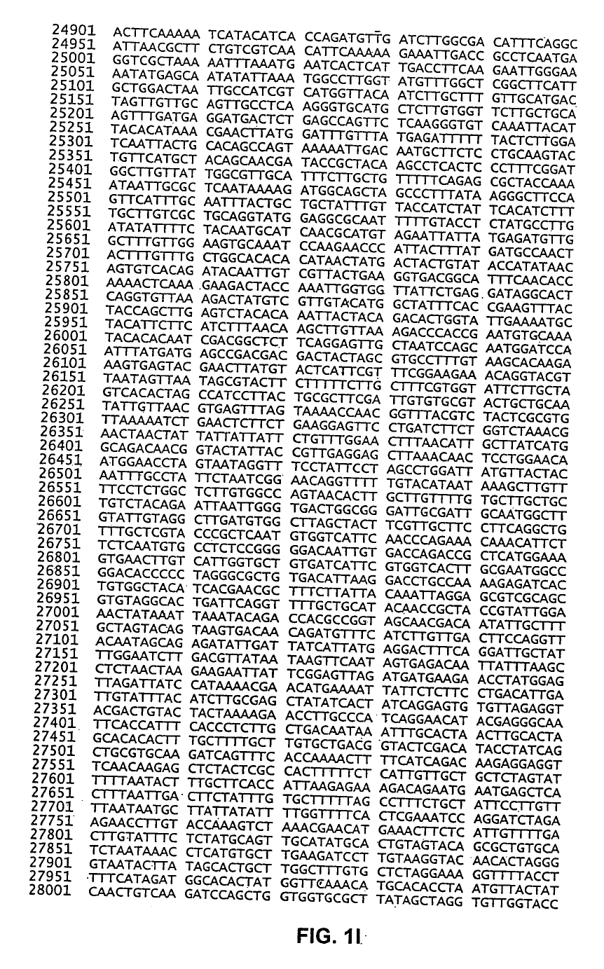

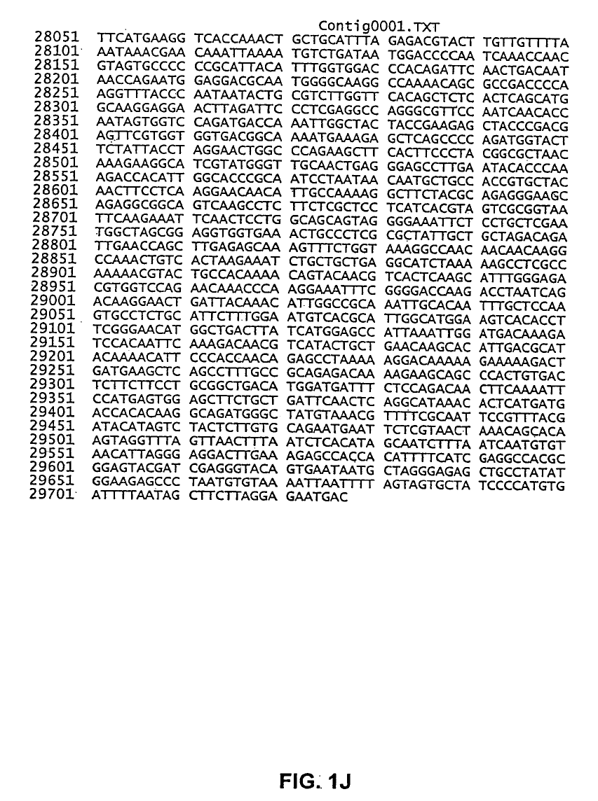

In a more preferred embodiment, the coronaviruses which can be treated, prevented, or managed by methods of the present invention are human pathogens. Examples of preferred methods relate to the treatment, prevention or management of viruses which include, but are not limited to, human respiratory coronavirus 229-E, human respiratory coronavirus OC43, human enteric corona virus, and severe acute respiratory syndrome (SARS)-associated coronavirus the genome of which is found in FIG. 1.

In another more preferred embodiment, coronaviruses which can be treated, prevented, or managed by methods of the present invention cause respiratory infections or diseases (i.e., respiratory coronaviruses), especially in humans. In a specific preferred embodiment, the respiratory coronavirus is SARS-associated coronavirus. The SARS-associated coronavirus to be treated, prevented, or managed by methods of the present invention can have the genomic sequence shown in FIG. 1 (also available at www.cdc.gov/ncidod/sars/sequence.htm) or Drosten et al., 2003, “Identification of a Novel Coronavirus in Patients with Severe Acute Respiratory Syndrome”, New England Journal of Medicine Apr. 10, 2003:epub ahead of print. Additionally, treatment, prevention, or management of different strains or variants of SARS-associated coronaviruses (either currently known or yet to be identified) are encompassed by the methods of the invention. In one embodiment, the SARS-associate coronavirus strain or variant is 75%, 80%, 85%, 90%, 95%, 98%, or 99% homologous to the SARS-associated coronavirus shown in FIG. 1. In another embodiment, the SARS-associate coronavirus strain or variant hybridizes to the nucleic acid sequence of the SARS-associated coronavirus shown in FIG. 1, or a fragment thereof e.g., an open reading frame, under high stringency.

In another preferred embodiment, the invention encompasses the treatment, prevention or management of coronavirus which does not infect the gastro-intestinal tract (i.e., is not a gastro-intestinal tract coronavirus).

The present invention also relates to methods of treating, preventing, or managing enveloped positive-sense single-stranded RNA viruses identifiable as phylogenetically corresponding or relating to the family Coronaviridae. Additionally, the enveloped positive-sense single-stranded RNA viruses to be treated, prevented, or managed by methods of the present invention encompass additional variants or strains of coronaviruses of yet to be identified (e.g., SARS-associated coronavirus strain that has a different sequence from the strain shown in FIG. 1 or in Drosten et al., 2003, “Identification of a Novel Coronavirus in Patients with Severe Acute Respiratory Syndrome”, New England Journal of Medicine Apr. 10, 2003:epub ahead of print).

A virus is identifiable as phylogenetically corresponding or relating to the family Coronaviridae using any technique known to the skilled artisan, e.g., phylogenetic analyses, sequence similarity, or genomic organization similarity.

In one embodiment, phylogenetic analyses can be used to identify a virus as phylogenetically corresponding or relating to the family Coronaviridae. Many methods or approaches are available to analyze phylogenetic relationship; these include distance, maximum likelihood, and maximum parsimony methods (Swofford, et. al., Phylogenetic Inference. In Molecular Systematics. Eds. Hillis, Mortiz, and Mable 1996. Sinauer Associates: Massachusetts, USA. pp. 407-514; Felsenstein, 1981, J. Mol. Evol. 17:368-376). In addition, bootstrapping techniques are an effective means of preparing and examining confidence intervals of resultant phylogenetic trees (Felsenstein, 1985, Evolution 29:783-791). Any method or approach using nucleotide or peptide sequence information to compare Coronaviridae isolates can be used to establish phylogenetic relationships, including, but not limited to, distance, maximum likelihood, and maximum parsimony methods or approaches. Any method known in the art can be used to analyze the quality of phylogenetic data, including but not limited to bootstrapping. Alignment of nucleotide or peptide sequence data for use in phylogenetic approaches, include but are not limited to, manual alignment, computer pairwise alignment, and computer multiple alignment. One skilled in the art would be familiar with the preferable alignment method or phylogenetic approach to be used based upon the information required and the time allowed.

In another embodiment, sequence similarity can be used to identify a virus as phylogenetically corresponding or relating to the family Coronaviridae. In a specific embodiment, two or more nucleic acids or amino acid sequences can be compared by BLAST (Altschul et al., 1990, J. Mol. Biol. 215:403-410) to determine their sequence similarities. BLAST comparisons can be performed using the Clustal W method (MacVector™). The alignment of two or more sequences by a computer program may or may not be followed by manual re-adjustment.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide comparisons can be performed with the NBLAST program. BLAST amino acid sequence comparisons can be performed with the XBLAST program. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table can be used. The gap length penalty can be set by the skilled artisan. The percent identity between two sequences can be determined using techniques similar to those described above, with or without allowing gaps. In calculating percent identity, typically only exact matches are counted.

In another specific embodiment, two or more nucleic acids can be compared by hybridization to determine sequence similarities. The nucleic acids are hybridized under conditions of high stringency. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×106 cpm of 32P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols, 1994-1997 John Wiley and Sons, Inc.).

In another embodiment, genomic organization can be used to identify a virus as phylogenetically corresponding or relating to the family Coronaviridae. For a representative genomic organization of a coronavirus se, e.g., Fields Virology 3^{rd} Edition, 1996, edited by Fields, Knipe, and Howley, Lippincott-Raven Publishers:Phoiladelphia, pages 1075-1120 and SARS-associated coronavirus strain that is different from the strain shown in Drosten et al., 2003, “Identification of a Novel Coronavirus in Patients with Severe Acute Respiratory Syndrome”, New England Journal of Medicine Apr. 10, 2003:epub ahead of print).

The present invention provides methods for introducing a therapeutically effective amount of a sulfated polysaccharide or combination of such sulfated polysaccharides into the blood stream, lymphatic system, and/or extracellular spaces of the tissue of a patient in the treatment and/or prevention of coronavirus infections, such as respiratory infections.

In a particular embodiment, the invention encompasses treatment, prevention or management of respiratory coronaviruses. In another preferred embodiment, the invention encompasses the treatment, prevention or management of coronavirus which is not of the gastro-intestinal tract.

Without being limited by theory, the sulfated polysaccharides of the invention have a high affinity for the lymph nodes thus have increased activity against viruses which populate or gestate in the lymphatic system. Thus, the present invention encompasses a method of administering a sulfated polysaccharide of the invention directly to or targeted for the lymphatic system of a patient.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of viral infection. These patients include, but are not limited to health care workers, e.g., doctors, nurses, hospice care givers; pilots; flight attendants; airline passengers; taxi drivers; bus drivers; train conductors; subway passengers; train passengers; military personnel; teachers; childcare workers; patients traveling to, or living in, foreign locales, in particular third world, East Asian, South-East Asian, or South Asian locales including social aid workers, missionaries, and foreign diplomats. The methods of the invention are particularly useful for healthy patients, i.e., those patients not displaying the symptoms of any other viral infection. Finally, the methods and compositions include the treatment of refractory patients or patients resistant to treatment such as resistance to reverse transcriptase inhibitors, protease inhibitors, etc.

In a particular embodiment, the methods of the invention are particularly useful to reduce the viral load in a patient already infected with a coronavirus. The methods of the invention are particularly useful to prevent death or other serious side effects associated with acute or initial infection with a coronavirus, particularly a respiratory coronavirus.

5.1.1 Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lie preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are susceptible to infection with the coronavirus to be treated, prevented, or managed (e.g. human embryonic kidney cells, human embryonic tracheal organ culture, human fibroblasts, intestinal cells such as MA-177 cells, rhabdomyosarcoma cells, fetal tonsil cells, vero cells such as vero E6 cells, and human embryonic intestinal organ culture or patient tissue samples) are grown in culture, and exposed to or otherwise administered a compound of the invention and the effect of the compound upon the cells is observed, e.g., ability of the cells to support coronavirus replication or infection. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a prophylactic or therapeutic dose of a sulfated polysaccharide of the invention or a pharmaceutically acceptable salt, solvate, hydrate, or stereoisomer thereof in the acute or chronic management of an infection or condition will vary with the nature and severity of the infection, and the route by which the active ingredient is administered. The dose, and perhaps the dose frequency, will also vary according to the infection to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. In general, the dose per day is in the range of from about 0.001 to 500 mg/kg, preferably about 0.01 to 200 mg/kg, more preferably about 0.005 to 100 mg/kg. For treatment of humans infected by coronavirus, about 0.1 mg to about 15 g per day is administered in about one to four divisions a day. Additionally, the recommended daily dose ran can be administered in cycles as single agents or in combination with other therapeutic agents. In one embodiment, the daily dose is administered in a single dose or in equally divided doses.

Different therapeutically effective amounts may be applicable for different infections, as will be readily known by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

5.1.2 Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, β-interferons, alkylating agents, hormones or cytokines.

The sulfated polysaccharides of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin Kg or Pen Vee K®)) or a quinolone (e.g., ofloxacin (Floxing), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefininox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The sulfated polysaccharides of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

The sulfated polysaccharides of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The sulfated polysaccharides of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, pleconaril, FUZEON™, didanosine, stavudine, and iamivudine.

The sulfated polysaccharides of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114 (IDEC)) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The sulfated polysaccharides of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-alpha, and IFN-gamma).

The sulfated polysaccharides of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The sulfated polysaccharides of the invention can be administered or formulated in combination with β-interferons which include, but are not limited to, interferon beta-1a and interferon beta-1b.

The sulfated polysaccharides of the invention can be administered or formulated in combination with an absorption enhancer, particularly those which target the lymphatic system, including, but not limited to sodium glycocholate; sodium caprate; N-lauryl-β-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancer can also be used. Thus, the invention also encompasses a pharmaceutical composition comprising one or more sulfated polysaccharides of the invention and one or more absorption enhancers.

The compounds of the invention and the other therapeutics agent can act additively or, more preferably, synergistically. In a preferred embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent.

5.2 Periodate Treated and Co-Charged Anionic Polysaccharides

The invention encompasses the use of sulfated polysaccharides that have been manipulated to reduce endocytosis by cell receptors and to increase the flexibility of the polysaccharide backbone to enable the efficient presentation of anionic charged groups to interact with regions on the targeted microbes.

One manipulation encompassed by the present invention is the treatment of sulfated polysaccharides with periodate. Periodate-treated anionic polysaccharides have increased flexibility due to periodate oxidation of some or all sugar residues. This treatment allows increased freedom of rotation and conformational flexibility of the polymers and provide flexible joints to facilitate biological interactions. Periodate-treated sulfated polysaccharides of the invention can have any counterion to ensure solubility including, but not limited to sodium, calcium, quaternary ammonium, and potassium. Thus, the invention encompasses the use of such compounds to treat or prevent coronavirus infections.

Materials which may be periodate treated and used within the methods and compositions described herein also include the polysaccharides of Table I below.

Other variations include the incorporation of non-sulfate groups, such as carboxymethyl groups and sulfonate groups. It should be recognized that modifying the charge of the sulfated polysaccharides described herein using e.g., carboxymethyl groups, may result in a material which has a lower percent of sulfur and yet will remain active in vivo. By lowering the degree of substitution of charge on the polysaccharide with either sulfonate or carboxymethyl groups, the ability of the polysaccharide to be endocyctosed by high charge receptors is greatly reduced, therefore increasing its plasma stability. Carboxymethyl dextran sulfate can be prepared using a modification of methods of preparation employed by others (McLaughlin and Hirbst, 1950, Can. J. Res. 28B:731-736; Brown et al. 1964, Arkiv. Kemi 22:189-206). Approximately 20 g of dextran is slurried in a mixture of isopropanol (350 ml) and 3.85M NaOH (40 ml) and is stirred for five minutes at 5° C. in a blender. Sodium chloroacetate (18 g) is added, and the whole mixture is stirred for 60 minutes at 5° C. under a nitrogen atmosphere, the mixture is removed from the blender and stored at 25° C. for three days. The degree of carboxymethyl substitution can be adjusted by varying the time at 25° C. from 1 day to 3 days as well as varying the mole ratio of ClCH₂COONa to anhydroglucose from 1 to 4 and keeping the mola ratio of ClCH₂COONa to NaOH to 1 to 1.4. After neutralization the sample is washed with 80% ethanol and dried.

5.3 Sulfated Polysaccharides for In Vivo Use

The invention encompasses the use of naturally occurring sulfated polysaccharides for administration in vivo preferably naturally occurring materials which have been modified to have a sulfation sufficient to eliminate or reduce binding of the sulfated polysaccharide by high charge density polyanion cell receptors and to provide antiviral activity to the sulfated polysaccharide. The sulfation range can be reached by chemical or enzymatic modification of natural or synthetic sulfated polysaccharide to achieve the desired percent of sulfation. Preferably, naturally occurring material can be modified chemically or enzymatically to the degree of sulfation range wherein the sulfation is effective to enable maximal interaction of constituent sulfate groups with the virus which causes the infection, and wherein the sulfated polysaccharide is not substantially endocytosed or degraded by cell receptor binding in the mammal, and thereby retains antimicrobial activity in vivo.

Listed in Table 1 below are examples of sulfated polysaccharides (not including dextran sulfate) which can be used, preferably after their degree of sulfation has been adjusted to treat or prevent coronavirus infections. It must be noted that flexible α-1,6-glycosidic compounds described above such as dextran sulfate or variants thereof are nevertheless preferred.

TABLE 1
Sulfated Polysaccharides

(14)-2-deoxy-2-sulfamido-3-O-sulfo-(14)-beta-D-glycopyranan
(derivative of chitosan)
2-acetamido-2-deoxy-3-O-sulfo(14)-beta-D-glycopyranan
(derivative of chitosan)
Achranthese bidentata polysaccharide sulfate
Aurintricarboxylic acid
Calcium spirulan
Carboxymethylchitin
Chemically degraded heparin (Org 31733)
Chondroitin polysulfate
Copolymer of sulphonic acid and biphenyl
disulphonic acid urea (MDL 10128)
Curdlan sulfate
Cyanovirin-N (from cyanobacterium)
Fucoidin
Galactan sulfate
Glucosamine-6-sulfate (monosaccharide)
Glycyrrhizin sulfate
Heparin
Inositol hexasulfate
Lentinan sulfate
Mannan sulfate
N-acylated heparin conjugates
N-carboxymethylchitosan-N,O-sulfate
Oligonucleotide-poly(L-lysine)-heparin complexes
Pentosan polysulfate (xylanopolyhydrogen sulfate)
Peptidoglycan DS-4152
Periodate degraded heparin
Phosphorothioate oligodeoxynucleotides
Polyacetal polysulfate
Polyinosinic-polycytidylic acid
Polysaccharides from Indocalamus tesselatus (bamboo leaves)
Prunellin
Rhamnan sulfate
Ribofuranan sulfate
Sodium lauryl sulfate
Sulfate dodecyl laminarapentaoside (alkyl oligosaccharide)
Sulfated bacterial glycosaminooglycan
Sulfated dodecyl laminari-oligomer (alkyl oligosaccharide)
Sulfated gangliosides
Sulfated laminara-oligosaccharide glycosides synthesized from
laminara-tetraose, laminara-pentaose, laminara-hexaose
Sulfated N-deacetylatedchitin
Sulfated octadecyl maltohexaoside (alkyl oligosaccharide)
Sulfated octadecyl ribofurnans
Sulfated oligoxylan (heparin mimetic)
Sulfated xylogalactans
Sulfatide (3′ sulfogalactosylceramide)
Sulfoevernan
Xylomannan sulfate

Each of sulfated polysaccharides listed above, as well as any other sulfated polysaccharide that has anti-viral activity in vitro, may be modified to bring their degree of sulfation or ionic charge to a level suitable for their use in the methods or compositions of the invention.

The invention further encompasses a method of treating, preventing or managing a viral infection in a mammal which comprises administering a compound chosen from the group consisting of cellulose sulfate; (14)-2-deoxy-2-sulfamido-3-O-sulfo-(14)-beta-D-glycopyranan (derivative of chitosan); 2-acetamido-2-deoxy-3-O-sulfo-(14)-beta-D-glycopyranan (derivative of chitosan); Achranthese bidentata polysaccharide sulfate; Aurintricarboxylic acid; Calcium spirulan; Carboxymethylchitin; Chemically degraded heparin (Org 31733); Chondroitin polysulfate; Copolymer of sulphonic acid and biphenyl disulphonic acid urea (MDL 10128); Curdlan sulfate; Cyanovirin-N (from cyanobacterium); Fucoidin; Galactan sulfate; Glucosamine-6-sulfate (monosaccharide); Glycyrrhizin sulfate; Heparin; Inositol hexasulfate; Lentinan sulfate; Mannan sulfate; N-acylated heparin conjugates; N-carboxymethylchitosan-N,O-sulfate; Oligonucleotide-poly(L-lysine)-heparin complexes; Pentosan polysulfate (xylanopolyhydrogen sulfate); Peptidoglycan DS-4152; Periodate degraded heparin; Phosphorothioate oligodeoxynucleotides; Polyacetal polysulfate; Polyinosinic-polycytidylic acid; Polysaccharides from Indocalamus tesselatus (bamboo leaves); Prunellin; Rhamnan sulfate; Ribofuranan sulfate; Sodium lauryl sulfate; Sulfate dodecyl laminarapentaoside (alkyl oligosaccharide); Sulfated bacterial glycosaminooglycan; Sulfated dodecyl laminari-oligomer (alkyl oligosaccharide); Sulfated gangliosides; Sulfated laminara-oligosaccharide glycosides synthesized from laminara-tetraose, laminara-pentaose, laminara-hexaose; Sulfated N-deacetylatedchitin; Sulfated octadecyl maltohexaoside (alkyl oligosaccharide); Sulfated octadecyl ribofurnans; Sulfated oligoxylan (heparin mimetic); Sulfated xylogalactans; Sulfatide (3′ sulfogalactosylceramide); Sulfoeveman; and Xylomannan sulfate, wherein the percent of sulfation of said compound has been controlled or modified to enable maximal interaction of constituent sulfate groups with the coronavirus causing the infection, and wherein the compound is not substantially endocytosed or degraded by cell receptor binding in the mammal, thereby retaining antiviral activity in vivo.

5.4 Pharmaceutical Compositions and Dosage Forms

Pharmaceutical compositions and single unit dosage forms comprising a sulfated polysaccharide of the invention, or a pharmaceutically acceptable salt, hydrate or stereoisomer thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, delivery via nebulizer, pulmonary delivery or topical administration. Pharmaceutical compositions and dosage forms of the invention typically also comprise one or more pharmaceutically acceptable excipients.

In an alternative embodiment, pharmaceutical composition encompassed by this embodiment include a sulfated polysaccharide of the invention, or a pharmaceutically acceptable salt, hydrate or stereoisomer thereof, and at least one additional therapeutic agent. Examples of additional therapeutic agents include, but are not limited to, those listed above.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington 's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise sulfated polysaccharides of the invention, or a pharmaceutically acceptable salt, hydrate, or stereoisomers thereof comprise 0.1 mg to 1500 mg per unit to provide doses of about 0.01 to 200 mg/kg per day.

5.4.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington 's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.4.2 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

5.4.4 Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.5 Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). In a preferred embodiment of the invention, the sulfated polysaccharides of the invention have a molecular weight greater than about 500,000 when administered topically.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

5.4.6 Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The sulfated polysaccharides of the invention may also be administered directly to the lung by inhalation. For administration by inhalation, a sulfated polysaccharide can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a sulfated polysaccharide directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a sulfated polysaccharide to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a sulfated polysaccharide to the lung is a liquid spray device supplied, for example, by Aradigm Corporation. Liquid spray systems use extremely small nozzle holes to aerosolize liquid drug formulations that can then be directly inhaled into the lung.

In a preferred embodiment, a nebulizer device is used to deliver sulfated polysaccharides to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In a particularly preferred embodiment, an electrohydrodynamic (“EHD”) aerosol device is used to deliver sulfated polysaccharides to the lung. EHD aerosol devices use electrical energy to aerosolize liquid drug solutions or suspensions (see, e.g., Noakes et al., U.S. Pat. No. 4,765,539; Coffee, U.S. Pat. No., 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the sulfated polysaccharides formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of sulfated polysaccharides will be known to the skilled artisan and are within the scope of the invention.

Liquid drug formulations suitable for use with nebulizers and liquid spray devices and EHD aerosol devices will typically include a sulfated polysaccharide with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of sulfated polysaccharide. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611, which are herein incorporated by reference) A sulfated polysaccharides can also be formulated in rectal or vaginal compositions such as suppositories or retention enemas, e.g., containing conventional suppository bases such as cocoa butter or other glycerides.

In addition to the formulations described previously, a sulfated polysaccharide can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver sulfated polysaccharides. Certain organic solvents such as dimethylsulfoxide can also be employed, although usually at the cost of greater toxicity. A sulfated polysaccharide can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 1983, 23, 61; see also Levy et al., Science, 1985, 228, 190; During et al., Ann. Neurol., 1989,25,351; Howard et al., 1989, J. Neurosurg. 71, 105). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g. Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.7 Condoms and Prophylactic Devices

In a preferred embodiment of the invention, the sulfated polysaccharide can be used as a coating for a condom or other prophylactic device. Similarly, the sulfated polysaccharide can be used as a coating for surgical instruments and protective devices such as rubber gloves, mouth guards, CPR aids and surgical masks. When a sulfated polysaccharide of the invention is used as a coating as described herein, it is preferred to have a molecular weight higher than 500,000. The methods of using the sulfated polysaccharides of the invention as a coating will be well known by the skilled artisan. Similar methods can be found in U.S. Pat. No. 4,869,270 which is incorporated herein by reference.

5.4.8 Disinfectants and Detergents

In one embodiment of the invention, the sulfated polysaccharides of the invention can be used to disinfect inanimate objects in hospitals, laboratories, lavatories, auditoriums, stadiums, convention centers, restaurants, fitness centers, subway terminals, bus terminals, airports, post offices, offices, sewage treatment facilities, sewers, water treatment facilities, pumping stations, automobiles, airplanes, trains, homes, lockers, and furniture to prevent the spread of viruses or disease.

Disinfectant compositions comprise one or more sulfated polysaccharides of the invention in the form of powders, pastes, concentrates, solutions, sprays, soaps, foams, gels, lotions, creams, mouthwashes, handwashes, pretreated towels, pretreated towelettes, pretreated cotton swabs, or pretreated pads.

In a specific embodiment, the sulfated polysaccharides of the invention can be used to disinfect biological fluid including, but not limited to blood, plasma, ova, sperm, or semen. The sulfated polysaccharides of the invention can be added directly to the biological fluid or coupled to a solid support, including, but not limited to plastic beads, glass beads, or filters which is placed in contact with the biological fluid.

5.4.9 Nutritional Products and Dietary Supplements

The sulfated polysaccharides may be incorporated into nutritional products including, but not limited to food compositions, over the counter, and dietary supplements. The sulfated polysaccharides may be added to various foods so as to be consumed simultaneously. As a food additive, the sulfated polysaccharides of the invention may be used in the same manner as conventional food additives, and thus, only needs to be mixed with other components to enhance the taste. Taste enhancement includes, but is not limited to, imparting to food a refreshingness, vitality, cleanness, fineness, or bracingness to the inherent taste of the food.

It will be recognized that dietary supplements may not use the same formulation ingredients or have the same sterile and other FDA requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements may take the form of tablets or capsules prepared by conventional means and optionally including other dietary supplements including vitamins, minerals, other herbal supplements, binding agents, fillers, lubricants, disintegrants, or wetting agents, as those discussed above. The tablets may be coated by methods well-known in the art. In a preferred embodiment, the dietary supplement may take the form of a capsule or powder to be dissolved in a liquid for oral consumption.

The amount of sulfated polysaccharides in a beverage or incorporated into a food product will depend on the kind of beverage, food and the desired effect. In general, a single serving comprises an amount of about 0.1% to about 50%, preferably of about 0.5% to about 20% of the food composition. More preferably a food product comprises sulfated polysaccharides in an amount of about 1% to about 10% by weight of the food composition.

Examples of food include, but are not limited to, confectionery such as sweets (candies, jellies, jams, etc.), gums, bean pastes, baked confectioneries or molded confectioneries (cookies, biscuits, etc.), steamed confectioneries, cacao or cacao products (chocolates and cocoa), frozen confectioneries (ice cream, ices, etc.), beverages (fruit juice, soft drinks, carbonated beverages), health drinks, health bars, and tea (green tea, black tea, etc.).

5.5 Assays and Animal Models

The sulfated polysaccharides, compositions and dosage forms of the invention can be tested in vitro and in vivo by a variety of methods known in the art to test antiviral activity against coronaviruses. See, for example, the methods discussed below or used in the examples. A number of assays may be employed in accordance with the present invention in order to determine the rate of growth of a coronavirus in a cell culture system, an animal model system or in a human subject. The assays described herein may be used to determine the growth characteristics of a coronavirus over time in the presence of a compound of the invention.

In one embodiment, a coronavirus and a compound of the invention are added to cells in culture that are susceptible to infection with the coronavirus (e.g. human embryonic kidney cells, human embryonic tracheal organ culture, human fibroblasts, intestinal cells such as MA-177 cells, rhabdomyosarcoma cells, fetal tonsil cells, vero cells such as vero E6 cells, and human embryonic intestinal organ culture). In one embodiment, the number of virally infected cells can be compared to the number of virally infected cells present when cells are incubated with the coronavirus alone (in the absence of a compound of the invention). Anti-viral activity of the compound of the invention is demonstrated by a decrease in the number of infected cells in the presence of the compound of the invention. Any method known in the art can be used to determine the number of infected cells including, but not limited to, visual/microscopic inspection for cytopathic effect of infection (e.g., cell rounding, refractive appearance, cell detachment, cell lysis, formation of multinucleated syncytia), viral titer, number of plaques, immunofluorescent staining or immunoblot (after cell lysis) using an antibody which immunospecifically recognizes the coronavirus to be assayed or detection of a coronavirus-specific nucleic acid (e.g., by in situ hybridization, or after cell lysis by Southern blot or RT-PCR analysis). In another embodiment, the number of viable cells can be compared to the number of viable cells present when cells are incubated with the coronavirus alone (in the absence of a compound of the invention). Anti-viral activity of the compound of the invention is demonstrated by an increase in the number of viable cells in the presence of the compound of the invention. Any method known in the art can be used to determine the number of viable cells including, but not limited to, a trypan blue assay. In a specific embodiment, the coronavirus and the compound of the invention are added to the cells at the same time. In another specific embodiment, the coronavirus is added to the cells before the compound of the invention. In another specific embodiment, the compound of the invention is added to the cells before the coronavirus.

In another embodiment, a coronavirus and a compound of the invention are administered to animal subjects susceptible to infection with the coronavirus. The incidence, severity, length, viral load, mortality rate of infection, etc. can be compared to the incidence, severity, length, viral load, mortality rate of infection, etc. observed when subjects are administered the coronavirus alone (in the absence of a compound of the invention). Anti-viral activity of the compound of the invention is demonstrated by a decrease in incidence, severity, length, viral load, mortality rate of infection, etc. in the presence of the compound of the invention. In a specific embodiment, the coronavirus and the compound of the invention are administered to the animal subject at the same time. In another specific embodiment, the coronavirus is administered to the animal subject before the compound of the invention. In another specific embodiment, the compound of the invention is administered to the animal subject before the coronavirus.

Any method known in the art can be used to determine anti-viral activity in a subject. The growth rate of the coronavirus can be tested by sampling biological fluids/clinical samples (e.g., nasal aspirate, throat swab, sputum, broncho-alveolar lavage, urine, saliva, blood, or serum) from animal subjects at multiple time points post-infection either in the presence or absence of a compound of the invention and measuring viral titer. In a specific embodiment, the viral titer is determined by obtaining biological fluids/clinical samples from an infected subject, preparing a serial dilution of the sample and infecting a monolayer of cells that are susceptible to infection with the virus (e.g. human embryonic kidney cells, human embryonic tracheal organ culture, human fibroblasts, intestinal cells such as MA-177 cells, rhabdomyosarcoma cells, fetal tonsil cells, vero cells such as vero E6 cells, human embryonic intestinal organ culture, and cells isolated from the subject) at a dilution of the virus that allows for the emergence of single plaques. The plaques can then be counted and the viral titer expressed as plaque forming units per milliliter of sample.

In another specific embodiment of the invention, the growth rate of a coronavirus in a subject is estimated by the titer of antibodies against the virus in the subject. Antibody serum titer can be determined by any method well-known in the art, for example, but not limited to, the amount of antibody or antibody fragment in serum samples can be quantitated by, e.g., ELISA (for discussion regarding ELISAs see, e.g., Ausubel et al., eds, 1994, Current Protocols in Molecular Biology, Vol I, John Wiley & Sons, Inc., New York at 11.2.1).

In other specific embodiments, the growth rate of a coronavirus in cell culture or in subject is determined by assessing the presence of virus in a sample using any method well-known in the art, for example, but not limited to, immunoassay (e.g., ELISA), immunofluorescent staining or immunoblot analysis using an antibody which immunospecifically recognizes the coronavirus to be assayed, or detection of a coronavirus-specific nucleic acid (e.g., by Southern blot or RT-PCR analysis, etc.).

Samples from subjects containing intact cells can be directly processed, whereas isolates without intact cells may or may not be first cultured on a permissive cell line (e.g. human embryonic kidney cells, human embryonic tracheal organ culture, human fibroblasts, intestinal cells such as MA-177 cells, rhabdomyosarcoma cells, fetal tonsil cells, vero cells such as vero E6 cells, and human embryonic intestinal organ culture). Cultured cell suspensions can be cleared by centrifugation at, e.g., 300×g for 5 minutes at room temperature, followed by a PBS, pH 7.4 (Ca⁺⁺ and Mg⁺⁺ free) wash under the same conditions. Cell pellets can be resuspended in a small volume of PBS for analysis. Primary clinical isolates containing intact cells can be mixed with PBS and centrifuged at 300×g for 5 minutes at room temperature. Mucus is removed from the interface with a sterile pipette tip and cell pellets can be washed once more with PBS under the same conditions. Pellets can then be resuspended in a small volume of PBS for analysis.

In another embodiment, a compound of the invention is administered to a human subject infected with a coronavirus. The incidence, severity, length, viral load, mortality rate of infection, etc. can be compared to the incidence, severity, length, viral load, mortality rate of infection, etc. observed in human subjects infected with the coronavirus in the absence of a compound of the invention or in the presence of a placebo. Anti-viral activity of the compound of the invention is demonstrated by a decrease in incidence, severity, length, viral load, mortality rate of infection, etc. in the presence of the compound of the invention. Any method known in the art can be used to determine anti-viral activity in a subject such as those described previously.

Additionally, in vivo activity of a sulfated polysaccharide can be determined by directly administering the compound to an animal or human subject, collecting biological fluids/clinical samples (e.g., nasal aspirate, throat swab, sputum, broncho-alveolar lavage, urine, saliva, blood, or serum) and testing the biological fluids/clinical samples for anti-viral activity (e.g., by addition to cells in culture in the presence of the coronavirus).

In general, in vivo stability can be determined by a variety of models known to the skilled artisan. In particular, in vivo stability can be determined by a kidney perfusion assay. For either type of analysis, the test compound may be labeled, for example with tritium. A kidney perfusion technique is described in detail in Tay et al. (Am. J. Physiol., (1991), 260: F549-F554). Briefly, rat kidneys, e.g., from male Sprague-Dawley rats, are perfused with 5% bovine serum albumin (BSA) in modified Krebs Henseleit buffer containing amino acids and continually gassed with 95% O₂-5% CO₂. Samples that have been perfused may be subjected to ion-exchange chromatography using, for example, a 19×1/cm² column of sepharose Q. Samples are applied to the column in 6 M urea, 0.05 M Tris, 0.005% (w/v) Chaps, pH 7.0, and eluted with a linear gradient of 0.15-2.5 M NaCl in the same buffer at a flow rate of 0.5 ml/minute. Recoveries using this technique are very good.

The foregoing has demonstrated the pertinent and important features of the present invention. One of skill in the art will be appreciate that numerous modifications and embodiments may be devised. Therefore, it is intended that the appended claims cover all such modifications and embodiments.

### 6. WORKING EXAMPLES

The following examples are for the purpose of illustration only and are not intended as limiting the scope of the invention.

### 6.1 Example 1

### Synthesis of a Sulfated Dextran Having a Sulfation of 9.5%

Dextran T20(average molecular weight 20,000) was dried in vacuo at 60° C. overnight. The dried compound (100 g) was dissolved in 640 ml formamide (FA). Chlorosulfonic acid (CSA) 80 ml was added to FA 200 ml at a maximum of 45° C. in a 3-necked flask, then cooled in ice-water. The amount of CSA determines the ultimate sulfation of the sulfated dextran (180 ml CSA to 200 ml FA yields approximately 17% sulfur). The CSA/FA mix was slowly added (over two hours) to the dextran at a temperature of 40° C. After all of the CSA/FA was added, the mixture was stirred for 15 minutes at a temperature of 45° C. The mixture was cooled to 25° C. and 28% NaOH was added slowly to give a pH 7.5-8.5 with a maximum temperature of 50° C. For the first precipitation, 3 L of ethanol were added with stirring. Stirring was stopped and the mixture was allowed to stand. The supernatant was decanted and the precipitate was redissolved in 1.5 L of water. For the second precipitation 1.5 L ethanol were added with stifling and then the mixture was allowed to stand for two hours. The supernatant was decanted and the precipitate was redissolved in 900 ml of water, to which 17 g NaCl was added. For the third precipitation 800 ml ethanol were added with stirring and the mixture was allowed to stand for two hours. The optical rotation-maximum was measured. The supernatant was decanted and the precipitate was redissolved in 500 ml water. 2.8 g Na₂HPO₄ and 2.6 g NaH₂PO₄ were added. For the final precipitation 5 L ethanol were added and the precipitate was filtered on a glass filter and dried in vacuo at 50° C.

### 6.2 Example 2

### Periodate Oxidation

Following the modified method of Smith degradation used by Sandy J D, Biochem J., 177: 569-574, 1979; chrondroitin sulfate (240 mg) was dissolved in 0.25M NaClO₄ (47 ml) at room temperature. 5 ml of 0.5 M NaIO₄ was added and KOH was used to adjust the mixture to pH 5. The reaction was allowed to proceed in the dark for 72 hours. The mixture was then dialysed in visking tubing to remove the periodate.

### 6.3 Example 3

### Introduction of Anionic Sulfur Groups to Carboxymethyl Dextran

6.3.1 Sulfated Form of Carboxymethyl Dextran (Average mw 20,000) with a Sulfur Content of 9.5%.

Carboxymethyl dextran (CMD) is dried in vacuo at 60° C. overnight. CMD (100 g) is dissolved in 640 ml formamide (FA). Chlorosulfonic acid (CSA) 80 ml is added to FA 200 ml at maximum of 45° C. in a 3-necked flask then cooled in ice-water. The amount of CSA will determine the ultimate sulfur content of CMD (180 ml OSA to 200 ml FA yields approx 17% sulfur). The CSA/FA mix is added slowly (over 2 hours) to CMD at a temperature of 40° C. After all is added the mixture is stirred for 15 minutes at a temperature of 45° C. The mixture is cooled to 25° C. and 28% NaOH is added slowly to give a pH 7.5-8.5 with a maximum temperature of 50° C. For the first precipitation, 3 L of ethanol is added with stirring. Supernatant is decanted and then residue is redissolved in 1.5 L of water. For the second precipitation 1.5 L ethanol is added with stirring and then allowed to stand for 2 hours. Supernatant is decanted and residue is redissolved in 900 ml of water and then added to 17 g NaCl. For the third precipitation 800 ml ethanol is add with stirring and allowed to stand for 2 hours. The optical rotation maximum should be 0.3. Supernatant is decanted and the residue is redissolved in 500 m water. Add 2.8 g Na₂HPO₄ and 2.6 g NaH₂PO₄. For the final precipitation 5 L ethanol is added and filtered on a glass filter and is dried in vacuo at 50° C.

6.3.2 Sulfonated Form of Carboxymethyl Dextran (Average Molecular Weight 20,000).

Step 1. Dissolve 5 g dextran in water. Add 100 mg borohydride stir at room temp. for 30 min.

Step 2. Add sodium hydroxide pellets (10 g) and stir until dissolved and then sulfonate (12 g).

Step 3. Heat at 70° C. for 7 h. After 3 hours add a further 3 g of sulphonate. Continue heating for 4 hours.

Step 4. Neutralise with 5M HCl to pH 7.5 (Total volume(T)=75 ml) and gradually add 200 ml ethanol with good stirring. Stop stirrer and stand 1 hour.

Step 5. Decant supernatant; redissolve in water (T=60 ml) and add 150 ml ethanol with good stirring. Stand 1 hour.

Step 6. Repeat as Step 5.

Step 7. Decant off the supernatant-redissolve the residue in 60 ml water and ppte in 600 ml ethanol. Some concentrated sodium chloride solution may be added to the mixture to aid precipitation.

Step 8. Filter and dry in vacuo. Yield approx. 6 g.

### 6.4 Example 4

### Maximum Tolerated Dose

The multiple toxicity dose (MTD) of DES6 was assessed in a series of experiments where groups of five rats were given 100 or 200 mg/kg doses of DES6 mw=500,000. Body weights and overall behavioral assessments were determined for five days after injection. There were no overt signs of toxicity as determined by observation and body weight measurements. Subsequently rats were given a 500 mg/kg injection and observed for a further five days also without signs of toxicity. Finally animals were given a dose of 850 mg/kg. Results are provided below in Table 2.

TABLE 2
MAXIMUM TOLERATED DOSE (MTD)

| | | Average body | |
|---|---|---|---|
| | | weight (n = 5) | S.D |
| 200 mg/kg | | | |
| Day | 1 | 277.4 | 15.9 |
| | 2 | 277.9 | 13.9 |
| | 3 | 288.9 | 14.9 |
| | 4 | 294.4 | 15.2 |
| | 5 | 296.0 | 22.3 |
| | 6 | 300.1 | 25.4 |
| 500 mg/kg | | | |
| Day | 7 | 328.6 | 21.9 |

The foregoing has demonstrated the pertinent and important features of the present invention. One of skill in the art will be appreciate that numerous modifications and embodiments may be devised. Therefore, it is intended that the appended claims cover all such modifications and embodiments.

1. A method of treating, preventing or managing a coronavirus infection in a mammal comprising administering to a mammal in need thereof a therapeutically effective amount of a sulfated polysaccharide.

2. The method of claim 1 wherein said sulfated polysaccharide has a percent of sulfur above 2% and below 20% with respect to the simple sugar residue.

3. The method of claim 2 wherein the percent of sulfur is above 3%.

4. The method of claim 3 wherein the percent of sulfur is above 6%.

5. The method of claim 4 wherein the percent of sulfur is above 9%.

6. The method of claims 3, 4 or 5 wherein the percent of sulfur is below 20%, below 17% or below 13%.

7. The method of claim 6 wherein the percent of sulfur is below 17%.

8. The method of claim 7 wherein the percent of sulfur is below 13%.

9. The method of claim 5 wherein the viral infection is coronavirus which causes or leads to sudden acute respiratory syndrome (SARS).

10. The method of claim 9 wherein the sulfated polysaccharide has a molecular weight from about 5,000 to about 1,000,000.

11. The method of claim 1 wherein the sulfated polysaccharide has a molecular weight greater than 25,000.

12. The method of claim 1 wherein the sulfated polysaccharide has a molecular weight greater than 40,000.

13. The method of claim 1 wherein the sulfated polysaccharide has a molecular weight greater then 500,000 and is administered topically.

14. The method of claim 1 wherein the sulfated polysaccharide comprises D-glucopyranose residues linked by α-1,6 linkages.

15. The method of claim 1 wherein the sulfated polysaccharide comprises L-glucopyranose residues.

16. The method of claim 1 wherein the sulfated polysaccharide is sulfated dextran.

17. The method of claim 1 wherein the sulfated polysaccharide is conventional dextran sulfate.

18. The method of claim 1 wherein the sulfated polysaccharide is dextrin sulfate, cyclodextrin or carrageenan.

19. A method of treating, or preventing or managing a respiratory infection or disease caused by a coronavirus in a mammal comprising administering to a mammal in need thereof a therapeutically or prophylactically acceptable amount of a sulfated polysaccharide.

20. The method of claim 19 wherein the sulfated polysaccharide has a percent of sulfation above 2% and below 20% with respect to the simple sugar residue.

21. The method of claim 19 wherein the molecular weight is above 5,000 to about 1,000,000.

22. The method of claim 19 wherein the molecular weight is above 25,000.

23. The method of claim 19 wherein the molecular weight is above 40,000.

24. The method of claim 19 wherein the percent of sulfur is above 3%.

25. The method of claim 24 wherein the percent of sulfur is above 6%.

26. The method of claim 25 wherein the percent of sulfur is above 9%.

27. The method of claim 24, 25, or 26 wherein the percent of sulfur is below 20%.

28. The method of claim 27 wherein the percent of sulfur is below 17%.

29. The method of claim 28 wherein the percent of sulfur is below 13%.

30. A method of treating, preventing or managing a coronavirus infection in a mammal which comprises administering to said mammal a therapeutically effective amount of a co-charged anionic polysaccharide wherein said co-charged anionic polysaccharide has a percent of sulfur which enables maximal interaction with the virus and which is not substantially endocytosed or degraded by cell receptor binding in the mammal.

31. The method of claim 30 wherein the co-charged anionic polysaccharide is co-charged with carboxymethyl groups, sulfonate groups, sulfate groups or combinations thereof.

32. The method of claim 31 wherein the co-charged anionic polysaccharide is co-charged with carboxymethyl groups.

33. The method of claim 32 wherein the co-charged anionic polysaccharide is carboxymethyl dextran sulfate or carboxymethyl cellulose.

34. The method of claim 1 wherein the coronavirus is a human respiratory coronavirus 229-E, human respiratory coronavirus OC43, human enteric coronavirus or strains thereof.

35. The method of claim 1 wherein the coronavirus is not a gastrointestinal coronavirus or feline infectious peritonitis virus.

36. The method of claim 1 wherein the coronavirus is the virus having the sequence of FIG. 1, or a sequence which has 90% or greater homology to the sequence of FIG. 1.

37. The method of claim 1 further comprising the administration of an additional therapeutic agent or an absorption enhancer.

38. The method of claim 1 wherein the therapeutically or prophylactically effective amount is from about 0.001 to 200 mg/kg per day.

39. The method of claim 38 wherein the therapeutically or prophylactically effective amount of the polysaccharide is from about 0.005 to 100 mg/kg per day.

40. The method of claim 1 wherein the therapeutically or prophylactically effective amount of the sulfated polysaccharide is from about 0.1 mg/kg/day to about 1,500 mg/kg/day.

41. The method of claim 1 wherein the mammal is a human.

42. The method of claim 1 wherein the therapeutically or prophylactically effective amount of the polysaccharide or is administered parenterally.

43. The method of claim 1 wherein the therapeutically or prophylactically effective amount of the polysaccharide is administered orally.

44. The method of claim 1 wherein the therapeutically or prophylactically effective amount of the polysaccharide is administered mucosally.

45. The method of claim 1 wherein the therapeutically or prophylactically effective amount of the polysaccharide is administered into the lung.

46. The method of claim 1 wherein the therapeutically or prophylactically effective amount of the polysaccharide is administered topically.

47. A pharmaceutical composition for treatment of coronavirus infection which comprises a therapeutically effective amount of a sulfated polysaccharide or a co-charged anionic polysaccharide having a percent of sulfur greater than 2% and less than 20%. wherein the amount is sufficient to neutralize said coronavirus and prevent viral replication.


SEQUENCE LISTING (1 sequence)

SEQ ID NO: 1 - DNA, 29727 bp, SARS-related coronavirus (Urbani strain)
ttattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagttcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgctactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagccttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgac